(19) United States
(12) Patent Application Publication
(10) Pub. No.: US 2023/0211134 A1
(43) Pub. Date: Jul. 6, 2023

(54) INFUSION CATHETER WITH A BALLOON HAVING A SINGLE LUMEN AND AN INTERNAL WIRE, AND USES THEREOF

(71) Applicant: MIVI Neuroscience, Inc., Eden Prairie, MN (US)

(72) Inventors: Matthew F. Ogle, Edina, MN (US); Richard C. Mattison, Zimmerman, MN (US); John Wainwright, Foothill Ranch, CA (US)

(21) Appl. No.: 17/565,678

(22) Filed: Dec. 30, 2021

(51) Int. Cl. A61M 25/10; A61B 17/22

(52) U.S. Cl. CPC A61M 25/1025; A61M 25/10186; A61B 17/22; A61M 2025/1081; A61M 2025/1052; A61M 2205/3344; A61M 2205/3306; A61M 2205/3334; A61M 2205/3375; A61M 2025/109; A61B 2017/22079

(57) ABSTRACT

Balloon/infusion catheters comprise internal corewires within a single lumen structure in which the corewire can slide relative to the catheter tube within limits, and the balloon is attached to the catheter tube on one end and to the sliding corewire on the other end. The lumen provides fluid to inflate the balloon and to infuse fluid into the vessel proximal to the balloon. The infusion ports can have a polymer valve to limit infusion to lumen pressures at which the balloon is appropriately inflated. The balloon/infusion catheter can have an integral flow meter near its proximal end. Corresponding methods for use of the balloon/infusion catheter are described, such as for the delivery of hydraulic forces when used in conjunction with an aspiration catheter.

### FIELD OF THE INVENTION

The invention relates to catheters suitable for use in a patient's vasculature, in particular the neurovasculature of the brain and peripheral vessels. The catheters generally comprise a balloon for temporarily sealing the vessel flow and one or more ports for infusing liquid into the vessel behind the balloon.

### BACKGROUND OF THE INVENTION

Blood clots can cause significant health risks, with potentially fatal outcomes. In particular, ischemic strokes can be caused by clots within a cerebral artery. The clots block blood flow, and the blocked blood flow can deprive brain tissue of its blood supply. The clots can be thrombus that forms locally or an embolus that migrated from another location to the place of vessel obstruction, which in either case can be referred to as thrombus clot while obstructing the vessel. To reduce the effects of the cut off in blood supply to the tissue, time is a significant factor, and it can be desirable to restore blood flow in a reduced period of time. The cerebral artery system is a highly branched system of blood vessels, which provide blood to the brain and are connected downstream to the interior carotid arteries. The cerebral arteries can be very circuitous. Medical treatment devices should be able to navigate along the circuitous route posed by the cerebral arteries for placement into the cerebral arteries. Aspiration has proven to be a useful procedure for clot removal alone or with other treatment modalities in cerebral as well as other arteries.

### SUMMARY OF THE INVENTION

In a first aspect, the invention relates to a balloon/infusion catheter comprising a corewire, a tube, a balloon and a proximal hub. The corewire generally comprises a shaft, a distal landing structure and a proximal lever. The tube can comprise a shaft with a single lumen, a proximal end, and a distal end, and one or more infusion ports near the distal end, in which the corewire extends through the tube and wherein a gap between the corewire and a wall of the tube allows fluid to flow through the lumen. The balloon can comprise a polymer sleeve in a sealing engagement at a first end with the tube and at a second end with the landing structure such that a sealed enclosure is formed spanning between the distal end of the tube and the landing structure. In general, the proximal hub is attached around the proximal end of the tube in a sealed configuration, and the proximal hub can comprise a wall, a corewire slide and a connector forming a continuous lumen through the hub extending the lumen of the tube, in which the corewire slide comprises a distal stop and a proximal stop that are configured to engage the proximal tab of the corewire at corresponding positions of the corewire relative to the tube to limit the range of relative positions of the corewire with the corewire free to slide within the tube between the proximal stop and the distal stop. The connector establishes a fluid channel from an opening into the connector to the interior of the balloon and to the one or more infusion ports.

The balloon/infusion catheter can be part of a system for clot removal from a bodily vessel further comprising a guide catheter and an aspiration catheter, in which the aspiration catheter can be configured to pass through the guide catheter with a distal section with an aspiration port extending distally from the guide catheter and in which the balloon infusion catheter can pass through the length of the aspiration catheter to have the balloon and one or more infusion ports extending distally from the aspiration catheter.

In a further aspect, the invention relates to a method for performing fluid infusion into a sealed off segment of a blood vessel, the method comprising guiding balloon/infusion catheter having a single lumen, without the aid of a guidewire from the carotid artery to a cerebral artery, inflating the balloon to seal off the blood vessel, in which the inflation comprises delivering fluid into the lumen; and infusing fluid proximal to the inflated balloon by delivering sufficient pressure of fluid into the lumen to release fluid through the polymer valve. The balloon/infusion catheter can comprise a tube with one or more infusion ports extending through the wall of the tube, a hub with a connector secured to the proximal end of the tube, a polymer balloon sealed on one side to the tube distal to the polymer valve, a corewire extending through the lumen of the tube, a landing structure secured to another end of the balloon such that a sealed enclosure is formed spanning the distal end of the tube and the landing structure, in which the landing structure is secured to the corewire, and slide limiter to limit the relative motion of the corewire relative to the tube, wherein during the guiding, the balloon is in a taut configuration over an opening into the lumen and wherein the hub is connected to a fluid source for inflating the balloon and providing infusion liquid;

In another aspect, the invention pertains to a balloon/infusion catheter comprising a corewire, a tube comprising a shaft with a single lumen, a proximal end, and a distal end, and one or more infusion ports near the distal end, a balloon having a sealed interior in fluid communication with the single lumen, a proximal hub, and a flow meter. The corewire generally extends through the tube and wherein a gap between the corewire and a wall of the tube allows fluid to flow through the lumen. The proximal hub is attached around the proximal end of the tube in a sealed configuration, and the proximal hub can comprise a wall and a connector forming a continuous lumen through the hub extending the lumen of the tube, in which the connector establishes a fluid channel from an opening into the connector to the interior of the balloon and to the one or more infusion ports. The flow meter can be configured to provide a value related to the flow rate through the single lumen.

### BRIEF DESCRIPTION OF THE DRAWINGS

FIG. 1 is a side view of a single lumen balloon/infusion catheter with an integrated distal coil.

FIG. 2 is a side view of a tubular shaft component of the single lumen balloon/infusion catheter of FIG. 1.

FIG. 3A is a side view of a core wire assembly of the single lumen balloon/infusion catheter of FIG. 1 prior to mounting of a distal coil.

FIG. 3B is a sectional view of the core wire assembly of FIG. 3A taken along line B-B of FIG. 3A.

FIG. 4A is a fragmentary, side view of the core wire assembly of FIG. 3 with an integrated distal coil.

FIG. 4B is a fragmentary side view of an embodiment of a corewire with an optical fiber extending through its core and connecting to the distal coil with an exposed region in the interior of the balloon.

FIG. 5A is a fragmentary, side view of the proximal end of a core wire.

FIG. 5B is a fragmentary, top view of the proximal end of a core wire.

FIG. 5C is a fragmentary, top view of a balloon/infusion catheter showing the hub having a slide where a portion of a lever portion of the corewire extends beyond the slide wall.

FIG. 5D is a fragmentary, sectional view of the slide portion of FIG. 5C taken along line D-D of FIG. 5C.

FIG. 5E is a fragmentary, side view of FIG. 5C rotated 90 degrees along the axis of the catheter.

FIG. 5F is a fragmentary, sectional view of the slide of FIG. 5E taken along line F-F of FIG. 5E.

FIG. 6A is a fragmentary, side view of the proximal end of an alternative embodiment of a corewire.

FIG. 6B is a fragmentary, top view of the proximal end of the corewire of FIG. 6A.

FIG. 6C is a fragmentary, top view of a balloon/infusion catheter showing the hub having a slide where a portion of a lever portion of the corewire is retained within the slide wall of the hub.

FIG. 6D is a fragmentary, sectional view of the slide of FIG. 6C taken along line D-D of FIG. 6C.

FIG. 7 is a view of a treatment system comprising a balloon/infusion catheter, a stent retriever and an aspiration system with a guide catheter and a cooperating aspiration catheter.

FIG. 8 is a separate view of the aspiration system of FIG. 7.

FIG. 9 is a side view of an alternative embodiment of an aspiration catheter.

FIG. 10 is a side view of an embodiment of a stent retriever.

FIG. 11 is a schematic diagram of a patient depicting delivery of a treatment system percutaneously with entry into a femoral artery.

FIG. 12 is a fragmentary view of a cerebral artery with a catheter delivered over a balloon/infusion catheter such that the distal end of the balloon/infusion catheter extends past the clot.

FIG. 13 is a fragmentary view depicting the cerebral artery of FIG. 12 in which the balloon of the balloon/infusion catheter is inflated.

FIG. 14 is a side view of a balloon/infusion catheter in which the balloon is deflated and the corewire is in a distal position within the slide.

FIG. 15 is a side view of a balloon/infusion catheter in which the balloon is inflated and the corewire is in a proximal position within the slide.

FIG. 16 is a fragmentary view depicting the cerebral artery of FIG. 13 in which hydraulic forces are established with aspiration and infusion resulting in the movement of thrombus from the clot in a proximal direction.

FIG. 17 is a fragmentary view depicting the cerebral artery of FIG. 16 in which thrombus from the clot has reached the aspiration opening of the aspiration catheter.

FIG. 18 is a fragmentary view depicting the cerebral artery of FIG. 17 in which the balloon is at least partially deflated and the catheter and balloon/infusion catheter are being removed from the vessel.

FIG. 19 is a fragmentary view depicting the cerebral artery of FIG. 13 in which a retrieval device is delivered through the catheter.

### DETAILED DESCRIPTION OF THE INVENTION

The use of hydraulic forces have been found to be a suitable approach to improve aspiration efficiency while providing reduced forces on a vessel in a patient, and to assist with fluid delivery, desirable devices are described with balloons configured for fluid infusion proximal to the balloon. The catheters have a single lumen that is used to inflate the balloon and to infuse liquid into the vessel. Generally, the catheter has a sliding internal wire and a polymer sleeve over an infusion port to function as a valve to allow infusion while maintaining sufficient balloon inflation. In some embodiments, a slide element associated with the wire can be controlled manually, and in alternative embodiments, the sliding of the guidewire can be controlled with fluid pressure where the wire can freely slide over a fixed range. The sliding wire, in combination with a floating distal tip, provides for improved control of the balloon status as well as rapid inflation and deflation of the balloon. A valved infusion port can limit the pressure of the balloon on the vessel wall. The catheter is designed for effective use with an aspiration catheter to establish hydraulic forces for clot removal, as well as optionally with other treatment components, such as a stent retriever. The catheter can be particularly effective for removal of clots from cerebral vessels to alleviate acute ischemic stroke events, but it can be effective to other procedures such as removal of clots in peripheral arteries or veins.

The infusing balloon catheter described herein can provide flushing of a vessel proximal to the balloon. As used herein, proximal is used as conventional in the art to refer to a direction in the vessel leading to the point of entry of the catheter into the vasculature, and distal refers to the direction in the vessel away from the point of entry of the catheter. The single lumen design of the catheter with a covered infusion port provides essentially for infusion to occur once the balloon is filled at a certain pressure such that fluid enters the vessel once it is occluded by the inflated balloon. This design of the catheter can be particularly effective for use in combination with an aspiration catheter such that hydraulic forces are established in the vessel, although the device can be used in other applications.

Applicant has described a related device in published U.S. patent application 2018/0098778 to Ogle (hereinafter the '778 application), entitled “Hydraulic Displacement and Removal of Thrombus Clots, and Catheter for Performing Hydraulic Displacement,” incorporated herein by reference. The present device is an improvement over embodiments of the catheter presented in the '778 application. In particular, the wire within the present catheter can slide over a prescribed range. The limited sliding feature provides for a convenient attachment of the balloon. Limits on the sliding avoid excessive forces on the balloon structure, and provides for more consistent inflation/deflation of the balloon. In some embodiments, manual control on wire sliding can provide additional control. With the sliding wire and the alternative balloon attachment, the balloon can be inflated and deflated relatively quickly, and the ability to have a balloon material with a convenient configuration with desirable pressure response characteristics. Also, the recent design allows for a relatively small profile device, that can be delivered like a guidewire in some embodiments.

The balloon can be formed from an elastomeric polymer with two loops or edges connected by a sealed tube of polymer forming the balloon. So the balloon material has the topology or configuration of a cylinder, although the unstrained shape may not be cylindrical since the sides may be curved in some embodiments. Each edge is sealed to enclose the interior of the balloon for inflation, with one end of the balloon sealed to the catheter body and the other end sealed to a floating distal tip. The floating distal tip is connected to the internal corewire. Inflation of the balloon results in the wire sliding in a proximal direction relative to the catheter hub. When liquid infusion is stopped and the balloon is deflated, the wire can move in a distal direction, which is facilitated by the balloon returning to is original configuration. In some embodiments, the slide extends through the wall of the slide hub to allow for manual activation. While various embodiments are possible to maintain fluid isolation, in one embodiment, a polymeric cover can seal the slot allowing for the manual slide function without loss of fluid or fluid pressure. For appropriate embodiments, an operator can then move the slide under the polymeric sheet.

Less invasive procedures are commonly performed in the vasculature using catheter based systems for reaching remote locations in a selected blood vessel for the performance of various treatment processes. These procedures can also be referred to as percutaneous procedures or transluminal procedures, in contrast with open surgical procedures, to emphasize the delivery through a vessel lumen. The discussion herein focuses on treatment of ischemic stroke since the devices can be particularly effective to treat these clinically important conditions, although the devices can be used in other procedures in the vasculature. Patients include humans and can include other mammals, such as pet animals and farm animals. Various methods have been developed for removal of clots within arteries, especially in the context of acute stroke. The procedures described herein provide an alternative to introduction of aspiration alone that can result in large pressure fluctuations within the vessel that also can injure blood vessel walls or other tissue adjacent the blood vessel. Also, use of aspiration without infusion tends to reduce local blood vessel pressure that can tend to reduce blood vessel diameter that can hinder removal of the clot by collapsing the vessel around the clot.

For the treatment of strokes, the treatment devices are advanced through arteries to blood vessels of the brain. Blood vessels generally relevant for acute stroke treatment are downstream in the blood flow from the internal carotid arteries, and arteries generally branch and decrease in average diameter as the vessel proceeds in a downstream direction in the arterial vasculature. The body has a right internal carotid artery and a left internal carotid artery. For convenience, the blood vessels downstream from the internal carotid arteries are referred to herein as cerebral arteries. The cerebral arteries can be accessed with catheter based systems from, for example, a femoral artery in the groin, an artery in the arm, or the carotid artery in the neck using hemostatic procedures and appropriate fittings, such as those known in the art. The cerebral arteries are known to follow circuitous paths, and complications in tracking devices along the vessels also follows due to shrinkage in diameter and branching of the vessels in a distal direction from the carotid artery as well as potentially dangerous conditions from damage to the blood vessel. It can be desirable to access tortuous narrow arteries for stroke treatment.

The basic aspects of some procedures described herein involve placement of an occlusion balloon distal to a clot, an infusion port between the occlusion balloon and the clot, and for providing hydraulic forces, an aspiration catheter with the suction opening proximal to the clot. The occlusion balloon can be compliant, i.e., formed of an elastic material, to occlude the vessel with modest forces on the vessel walls.

The infusion of liquid along with aspiration can generate a flow of liquid from distal to proximal of the clot with the corresponding generation of hydraulic forces and/or hydrodynamic forces. For convenience, herein the forces generated in the procedures are referred to as hydraulic forces, which in some sense, may include hydraulic forces, hydrodynamic forces or a combination thereof. Either by initiating the infusion prior to the aspiration and/or by controlling the various flow rates, the local pressure in the vessel may increase somewhat, and such a modest increase in pressure can flex the vessel wall to potentially loosen the grip of the vessel wall on the clot, although such vessel dilation is not necessarily performed. Thus, both the balloon expansion and the control of the pressures can work to loosen the clot to facilitate removal of the clot. The forces on the vessel wall can be controlled to reduce risk of injury to the vessel wall. The distal to proximal liquid flow correspondingly applies distal to proximal forces on the clot to tend to move the clot toward the aspiration catheter. The objective is to dislodge the clot so that it can be taken up by the aspiration catheter or to bring the clot to the aspiration opening for removal from the patient with the catheter.

For use in the neuro-vasculature, it may be desirable to use an independent guidewire to facilitate reaching more remote sites in the vessel, although a guidewire may be removed once additional components, such as a microcatheter or aspiration catheter, are delivered near the treatment site. The balloon/infusion catheter described herein is designed for possible delivery as a guidewire itself, so that a separate guidewire may not be used. To guide the procedures, generally, a guide catheter is placed in the carotid arteries, e.g., an internal carotid artery or a common carotid artery, through which additional devices can be guided to smaller vessels downstream. The guide catheter can comprise a hemostatic valve outside of the patient for the introduction of additional devices. In some embodiments, the guide catheter can also have an occlusion balloon that can be actuated to close off flow past the balloon. It can be desirable to stop flow for at least a part of the procedure. Also, aspiration can optionally be applied through the guide catheter as an alternative to or in addition to use of a separate aspiration catheter.

The use of suction alone has provided effective results for the treatment of acute ischemic stroke. Alternative devices are available to mechanically dislodge clots resulting in strokes, and some of these devices can be termed stent retrievers, although some of these devices are not quite derivatives of stents. Aspiration can also be used with stent retrievers or the like to provide the combined efficacies of the approaches. Furthermore, aspiration, while potentially very effective, the degree of aspiration provided for effective clot removal can deplete sections of the vessel of blood resulting in significant collapsing forces on the vessel and corresponding forces on adjacent tissue, which may be undesirable in some cases. While aspiration can be effective to dislodge and remove the clots, collapse of the vessel around the clot due to liquid removal may not facilitate the process and may increase the corresponding forces. The hydraulic/hydrodynamic forces described herein generate forces on both sides of the clot tending to remove the clot while potentially tending to dilate the vessel to also loosen the clot. Thus, the procedures can be designed to reduce the force extremes within the vessel and on the surrounding tissue while potentially being even more effective to remove clots.

Aspiration catheters are available to provide suction/aspiration for the hydraulic assisted procedures described herein, and these catheters are described in detail below. Aspiration catheters can be effectively used along with the balloon/infusion catheters to provide occlusion and infusion and optionally along with additional treatment devices, such as mechanical devices to engage clots and/or filter style devices that can capture loose emboli as well as optionally engaging the clot with a more cushioned element to limit forces on the vessel wall. Various systems for clot treatment are described below. Whether used to provide the hydraulic treatment alone or used in combination with .additional treatment or protection devices, the hydraulic treatment procedures described herein provide important tools with the possibility to provide more gentile removal of clots from vessels that can be particularly effective for the alleviation of acute ischemic stroke conditions.

### Catheters for Providing Occlusion and Proximal Infusion

Specific catheter structures are described for providing occlusion of distal flow with a balloon and proximal liquid delivery to flush the vessel proximal to the balloon. When used in combination with an aspiration catheter, the catheter can be used to help to generate hydraulic forces in the vessel against a clot in a distal to proximal direction. The catheter has a specific design to allow for delivery into a small vessel to provide the desired flows. Specifically, the catheters generally have a single lumen that provides for both balloon inflation and fluid infusion. A sliding corewire provides desires flexibility to the catheter. The corewire comprises a landing section near its distal end that provides structure for attaching the distal edge of the balloon. A distal coil or the like can extend from the corewire to form a distal tip of the catheter. The balloon and distal end of the corewire extend from the tubular portion of the catheter. The proximal edge of the balloon can be attached to the tubular body of the catheter to form an enclosed interior of the balloon in fluid communication with the catheter lumen, which terminates and directly opens into the interior of the balloon, generally with an annular opening. The corewire with the distal coil and the landing area for the distal end of the balloon can slide over a limited range relative to the tubular shaft forming most of the catheter body. Movement of the sliding corewire can transition the balloon from a configuration with the distal and proximal edges pulled apart and a configuration with the distal and proximal edges of the balloon moved together to provide for the balloon expansion. A proximal fitting can comprise a hub with a connector and a slide structure adjacent the connector, in which the slide structure engages the proximal end of the corewire to limit the extent of sliding. In some embodiments, the slide element can provide for manual sliding of the corewire, which can supplement or replace control of sliding using fluid pressure.

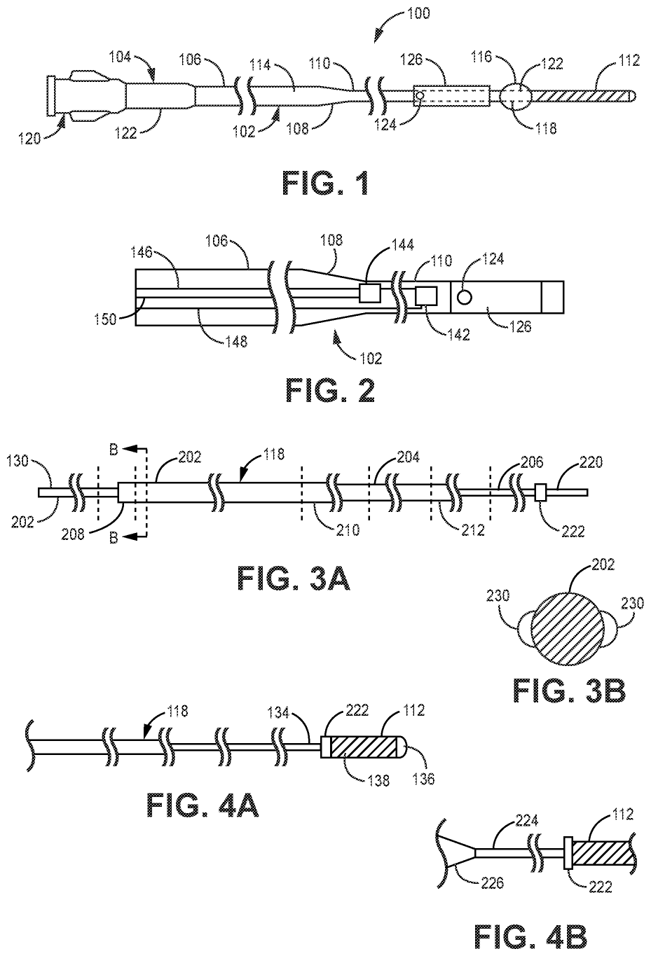

A desirable embodiment is shown in the figure, and the following discussion focuses on this embodiment. Referring to FIG. 1, a single lumen occlusion balloon/infusion catheter 100 comprises tubular shaft 102 forming lumen 114, balloon 116, corewire 118 floating within lumen 114 and extending from a distal end of tubular shaft 112, and a proximal fitting 104. Referring to the embodiment of FIGS. 1 and 2, tubular shaft 102 comprises a proximal section 106, distal section 110 with a smaller diameter than proximal section 106 and a transition section 108 connecting proximal section 106 and distal section 110. Transition section 108 can have an abrupt diameter change or a gradual transition of diameter, such as a linear transition. Proximal section 106 connects with proximal fitting 104. In alternative embodiments, tubular shaft 102 can have a constant outer diameter, or more than two sections with differing constant diameters and corresponding connecting sections. Balloon 116 can be a compliant balloon and has an interior in fluid communication with lumen 114 such that adjustment of fluid pressure within lumen 114 can expand or deflate balloon 116. Corewire 118 internal to the catheter can provide characteristics similar to a guidewire for the catheter, and a distal coil tip can be optional as long as the catheter tip is configured to avoid injury to the vessel wall.

Tubular shaft 102 comprises a lumen 114 extending from proximal fitting 104 to balloon 116. Lumen 114 opens at the distal end of tubular shaft 102 into the interior of balloon 116, such that fluid communication is established between lumen 114 and the interior of balloon 116. One or more infusion port(s) 124 can provide for infusion of liquid from lumen 114 delivered from a liquid source attached to proximal fitting 120 into the patient's vessel. Flow of liquid out from lumen 114 tends to lower the pressure within the lumen. Liquid pressure within lumen 114 also provides for inflation of balloon 116. Also, an embodiment of the infusion structure is shown in which an elastic cover 126, configured as a polymer valve, covers infusion ports 124. Elastic cover 126 can provide an added measure of control over the infusion process in which a certain amount of pressure may be applied to expand elastic cover 126 to provide for infusion. Elastic cover 126 can be sealed along one edge to tubular shaft 112 and open at an opposite edge to provide for the infusion. Elastic cover 126 can be made from the same or similar material as balloon, 116, although as long as elastic cover 126 provides desired elastic properties, it can be made from a distinct material.

Elastic cover 126 functioning as a polymer valve can be a tubular piece of elastomer sealed on one edge that opens as a check valve when the pressure is sufficient. Of course, non-tubular polymer sheets can be sealed along multiple edges with an open edge to provide a similar effect. The valve pressure response can be designed through the polymer and thickness, the length of the polymer tube relative to the sealed edge, with a longer tube eliciting a greater pressure to open the valve, and/or the any tension in the polymer, such as stretching the polymer over the catheter tube, if the relaxed polymer tube has a smaller diameter than the catheter tube. These design parameters can be adjusted to provide for e polymer valve to open at an appropriate pressure to expand the balloon. The balloon can be preconditioned by inflating and deflating the balloon during manufacturing. The preconditioned balloon can more easily inflate during a procedure. The balloon and polymer valve can be cut form a common extruded elastomer tube with the other design parameters used to adjust their responses during the procedure, although the properties can be adjusted to be different for the two components. In some embodiments, the elastic cover/polymer valve 126 can have a length from about 0.1 inches to about 2 inches, and in further embodiments from about 0.15 inches to about 1.5 inches. The balloon can have an unexpanded length from about 0.1 inches about 1 inch. A person of ordinary skill in the art will recognize that additional ranges of polymer element lengths along the catheter within the explicit ranges above are contemplated and are within the present disclosure.

The hydrodynamics should be balanced so that appropriate pressures to maintain the inflated balloon while correspondingly to provide for a desired infusion flow rate. Infusion port(s) 124 and elastic cover 126, if present, can be designed accordingly, and infusion port(s) 124 are also in appropriate proximity to balloon 156 to facilitate placement distal to the clot within tortuous vessels. In particular, the size and number of infusion port(s) 124 can be selected to provide appropriate infusion at pressures inflating balloon 116. In some embodiments, the farthest edge of an infusion port is no more than 5 centimeters from the closest edge of the balloon, and in further embodiments no more than 2.5 centimeters, and this spacing is general for all of the catheter embodiments in this section. A person of ordinary skill in the art will recognize that additional values of infusion port spacing within the explicit ranges above are contemplated and are within the present disclosure.

To facilitate monitoring of the pressures and flows through the system, the balloon/infusion catheter can be instrumented with sensors. One embodiment would be using an adjustable pressure infusion syringe pump or pressure bag or other infusion pump to provide a controllable volume and/or flow rate for the infusion. Various pressure infusion syringe devices are commercially available, such as Masterflex® Infusion Pump, Medfusion® Wireless Syringe Infusion Pump, a Merit Medical Pressure Infusor Bag™, ASP Medical Pressure Infusor Bags, and the like. Other pumps can also be adapted for these purposes as desired. This would allow the infusion pressure to be set and maintained. Referring to FIG. 2, tubular shaft 102 can be provided with one or more pressure sensors. Pressure sensor 142 is positioned to measure the pressure between the balloon and the original position of a clot, and pressure sensor 144 is positioned to measure the pressure between the original position of the clot and an aspiration catheter. Wiring for the sensors can be embedded within a polymer wall or otherwise tracked along the length of the tubular shaft to an exterior location. For example, as shown in FIG. 2, ground wire 146 connects to pressure sensor 142, 144, and wires 148, 150 connect to pressure sensors 142, 144 respectively. Various suitable pressure sensors can be used. Integrated circuit pressure sensors can be used such as the Infineon KP236 pressure sensor. A piezo resistive pressure die P330W is available from Nova®Sensor with a thickness of 120 microns. The use of these pressure sensors in a catheter is described in published U.S. patent application 2018/0010974A to Bueche et al., entitled “Pressure Sensor System,” incorporated herein by reference. Optical pressure sensors can also be used in all the various portions of the device similar to the Boston Scientific Comet II Guidewire. The pressure readings could also be transmitted wirelessly like the Abbott Pressurewire X guidewire.

Balloon 116 can be a tubular/cylindrical section of polymer that is connected at both ends to isolate the interior, although other topologically equivalent structures can be used to secure the balloon and isolate the interior. One edge of the balloon can be secured around tubular shaft 112 and the other edge can be secured around the corewire. The edges of the balloon can be secured using heat bonding, adhesive bonding, fastening with a band, combinations thereof, or the like. The fastening of the balloon to tubular shaft 112 at one end and floating corewire 118 at the other end advantageously permits balloon 116 to neck down when the catheter is advanced proximally or distally within the vessel, allowing the catheter system to more easily maneuver through the vasculature. Sliding of corewire 118 further permits rapid inflation or deflation of balloon 116 through the combined effect of fluid pressure and mechanical movement of the secured edges of the balloon relative to each other.

Referring to FIGS. 3 and 4, corewire comprises a proximal flattened section 130, a distal coil 110, and wire element 134. FIG. 3A depicts the corwire prior to mounting of the distal coil, and FIG. 4 depicts the distal section of the corewire with distal coil 112 mounted in place. A distal solder tip 136 provides a blunt tip and assist with holding distal coil 132 in place. Distal coil 112 is further connected to wire element 134 at a proximal solder joint 138 that in some embodiments can extend at or near the proximal end of the coil for from about 0.5% to about 25% of the coil length. Distal coil 112 should be broadly considered to cover analogous elements, such as a laser cut section of metal tubing cut to mimic a coil or the like. Flattened section 103 generally comprises a lever, as described further below. A proximal lever can engage with a slide element of the proximal fitting to limit the movement of corewire 118 relative to tubular shaft 110.

Referring to FIG. 3, corewire 118 comprises along its length flattened section 130, a first section 202, a second section 204, a third section 206, and tapered sections 208, 210, and 212, respectively connecting flattened section 130 with first section 202, first section 202 with second section 204 and second section 204 with third section 206. A fewer or greater number of tapered sections can be used, such as one, two, three, four, five or six. Sections of corewire 118 can have approximately discontinuous diameter changes or a desired gradual taper connecting adjacent sections of tapered sections. A majority of the length of corewire 118 can be provided by first section 202. Referring to FIG. 3B, first section 202 or a selected portion thereof can comprise wings 230 that can facilitate maintaining corewire 118 more centered within tubular shaft 102. Referring to FIG. 3A, third section 208 of the corewire body has a distal portion 220 with a somewhat larger diameter for mounting at least the proximal end of distal coil 112 and for forming solder joint 138, and landing element 222 for mounting the distal edge of balloon 116 to corewire 118. FIG. 4 shows corewire 118 with a distal end secured to coil tip 110, for example, with solder joint 138 and solder ball 136. All or portions of corewire 118 may be coated. For example, corewire 118 may be coated with a thermoplastic polymer such as polytetrafluoroethylene (PTFE) to facilitate sliding.

In some embodiments, corewire 118 can comprise a pressure sensor for measuring the pressure within the balloon. For such a configuration, an optical fiber based pressure sensor can be adapted for this application. A Bragg grating can be incorporated into the optical fiber for this purpose. The optical fiber can be placed in the interior of the corewire such that it is exposed in the region adjacent the balloon. Referring to FIG. 4B, optical fiber 224 exits a proximal adjacent portion of corewire 226 with landing element 222 and distal coil 112 at its distal side. Optical fiber based pressure sensors are described in U.S. Pat. No. 9,782,129B to Radman, entitled “Pressure Sensing Guidewires,” and published PCT application 2020/115211 to Stoker et al., entitled “Biomedical Pressure Sensor,” both of which are incorporated herein by reference. Also catheter based pressure sensors are available commercially from Millar, Houston, Tex., USA.

First section 202 of corewire 118 can have a diameter from about 0.007 in to about 0.012 in. Third section 208 can have a diameter from about 0.0015 in to about 0.005 in. Landing element 222 can have a diameter closer to the first section of the corewire. Second section 204 generally has a diameter intermediate between the first section and the second section, although second section diameter can be closer to the diameter of first section 202. Distal section 220 for mounting distal coil and forming the solder bond can have a length approximately corresponding to the length of the solder joint and a diameter from about 0.0025 to about 0.006 inches. Tapered section 212 can have a relatively significant length, such as 5 inches to 12 inches, as desired, and the balloon is generally attached in the extended uninflated configuration over at least a portion of this section. Tapered section 208 generally is short, such as an inch or less. Tapered section 210 can have an intermediate length, such as one inch to 8 inches. The distal coil can have a diameter generally over the same range as the widest section of the corewire and a length from about 0.2 to about 2 inches. A person of ordinary skill in the art will recognize that additional ranges of corewire dimensions within the explicit ranges above are contemplated and are within the present disclosure.

Along the length of the balloon/infusion catheter 102, the difference between the fixed wire diameter and the catheter inner diameter provides a gap for liquid flow that provides both control of balloon inflation and infusion. The gap (inner tube diameter minus the corewire diameter) may not be constant over the length. The gap along the proximal section of corewire 118 can be from about 0.003 in and about 0.007 in, and in some embodiments the proximal gap can be from about 0.0045 in to about 0.006 in. The gap at the distal most section of corewire 118 can be from about 0.006 in to about 0.0095 in, and in some embodiments from about 0.0075 in to about 0.009 in. Balloon 116 and infusion ports 124 can be located along the section of the distal fixed wire, and a larger gap can be desirable in view of the flows in this region. An intermediate region along the length of the catheter can have a narrowed gap. A person of ordinary skill in the art will recognize that additional ranges of diameters and gaps within the explicit ranges above are contemplated and are within the present disclosure.

Referring to FIG. 2, in some embodiments, the length of tubular shaft 102 can be from 150 cm to 250 cm and a particular embodiment based on delivery from a femoral artery can have a length from about 170 cm to about 200 cm. Distal segment 402 can have a length from about 15 cm to about 50 cm, and the taper segment 404 can have a length from about 1 cm to about 35 cm, although other values can be suitable. Catheter body 102 can have a wall thickness (materials and processing describe further below) from about 0.0015 inch (in) to about 0.005 in, so that the inner diameter is about twice the wall thickness less than the outer diameter. The proximal outer diameter can be from about 0.03 in to about 0.015, and the distal outer diameter can be from about 0.024 in to about 0.012. The inner diameters follow from the outer diameters and wall thicknesses. In embodiments, proximal segment 106 may have a thicker wall than distal segment 110 to provide greater pushability. A person of ordinary skill in the art will recognize that additional ranges of tubular shaft dimensions within the explicit ranges above are contemplated and are within the present disclosure. In a prototype embodiment for tubular shaft 102, 72D PEBAX tubing has a proximal segment with an OD of 0.021 inches and ID of 0.015 inches for a wall thickness of 0.003 inches, and distal segment 402 has a length of 20 cm, an OD of 0.019 inches, an ID of 0.014 inches, and a wall thickness of 0.0025 inches, which is formed through a bump extrusion process.

Referring to FIG. 1, proximal fitting or hub 104 comprises a connector 120 and a corewire slide 122. Connector 120 generally is used to connect the catheter to further fittings to attach a fluid source and possibly other components. Fittings 104 can comprise one integral structure or multiple elements, such as separate slide and connector components, that may be secured together with adhesive, thermal bonding and/or other bonding approaches to form a fluid tight seal.

Corewire slide 122 extends between connector 120 and tubular shaft 112. A proximal end of corewire 118 may be retained within corewire slide 122. In general, suitable connectors are known in the art for connector 120. In particular, connector 120 can be a Luer fitting, such as female Luer fittings, Tuohy-Borst connectors, or the like. Luer connectors or Tuohy-Borst connectors can be useful for attachment of standard or proprietary fittings or manifold, such as Y-branch fittings or the like to provide desired access to the lumen.

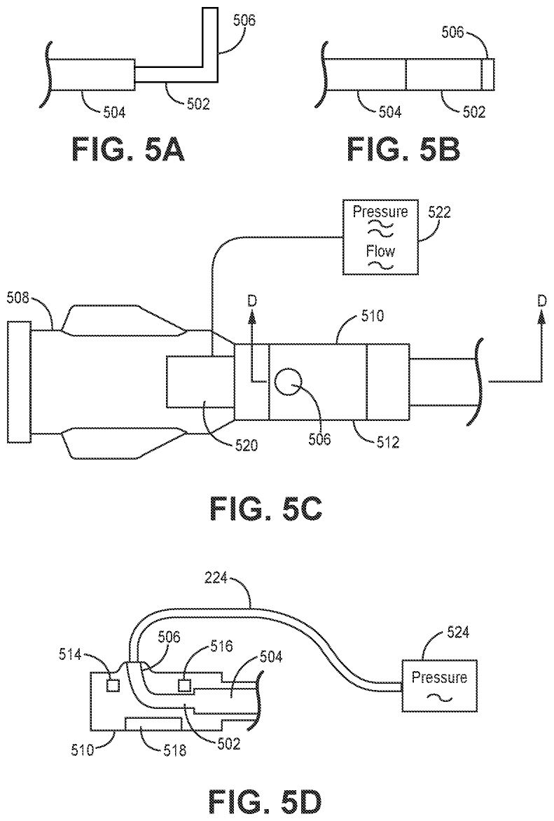

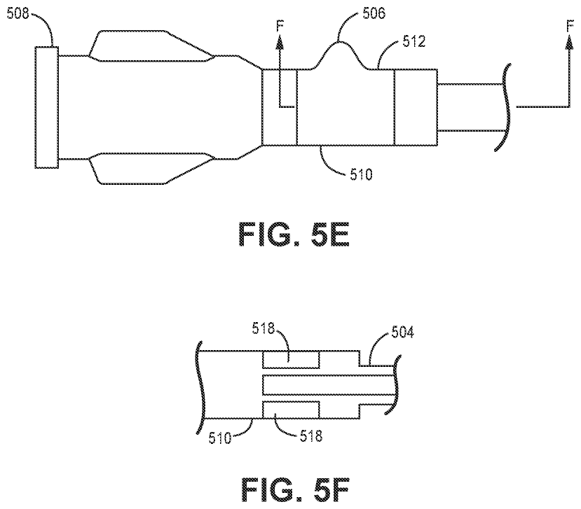

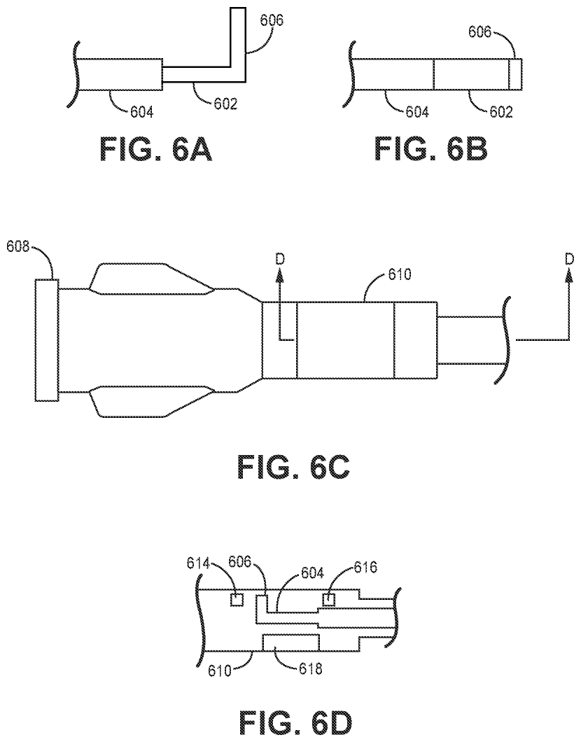

Two different embodiments for corewire slide 122 are depicted in FIGS. 5 and 6, respectively. The corewire slide in FIGS. 5A-F has a lever for manual engagement extending outward from the catheter hub wall. Referring to a corewire slide in FIGS. 5A and 5B, proximal section 502 of a corewire 504 may have a bent portion, or lever, 506. Bent portion 506 may be used to restrict the distance corewire 504 may slide in the proximal or distal directions within the treatment system. In particular, corewire slide 506 may have features, such as a plurality of tabs, described below, to limit the range of motion of corewire 504. Tabs should not interfere significantly with respect to fluid flow through the lumen.

Referring to FIGS. 5C and 5E, top and side views of hub 508 and slide 510 depict an embodiment where corewire 504 may be manually manipulated. Bent portion 506 may project radially beyond a wall of slide 510 such that a user may exert force on corewire 504 by pushing or pulling the bent portion 506. Bent portion 506 and slide 510 may be wrapped in a sleeve 512 preventing fluid from escaping through the wall of slide 510. For example, sleeve 512 may be a polymer film with sufficient mechanical integrity to avoid tearing or expansion in response to fluid pressures. As depicted in corresponding sectional views in FIGS. 5D and 5F, tabs 514, 516 limit the lateral movement of corewire 504 during manipulation. Additional tab(s) 518 may be used to maintain a generally central position of corewire 504 within slide 508. Tabs 514, 516, 518 can be metal or plastic elements that are bonded to or molded with the inner walls of the hub. It can be desirable to also integrate a flow meter in hub 508 to provide for measurement of flow in real time, and the flow measurement can be incorporated into a system control feature to coordinate the various operations of the system. Referring to FIG. 5C, flow meter 520 can be incorporated into the structure of hub 508. Flow meter 520 can be based, for example, on an optical fiber Bragg grating in combination with an LED element, as described in Ruiz-Vargas et al., entitled “Optical Flow Sensor for Continuous Invasive Measurement of Blood Flow Velocity,” Journal of Biophotonics Vol. 12(10), October 2019, e201900139, incorporated herein by reference. An alternative example of a flow meter would be a Doppler ultrasound sensor placed near the distal end of the catheter on the hub or adjacent the hub with suitable sensors that can be adapted for this as described in Cannata et al., “Development of a Flexible Implantable Sensor for Postoperative Monitoring of Blood Flow,” Journal of Ultrasound in Medicine, November 2012, Vol. 31(11) pp 1795-1802, incorporated herein by reference.

Hub 508 can be electrically connected to a display/controller that can display flow readings from flow meter 520 and/or pressure readings, such as from pressure sensors 142, 144. Referring to FIG. 5D, optical fiber 224 can exit slide 510 in a sealed configuration for connection to controller 524. Optical fiber 224 can be flexible, which is consistent with the sliding function of corewire 504. Controller 524 can provide the optical signal and analysis to allow for the pressure measurement, and controller 524 can comprise a display for the pressure measurement, or controller 524 can be interfaced with display 522 or a separate display to show the corresponding pressure value.

FIGS. 6A-6D illustrate an alternate embodiment of a slide 610 having a corewire 604 that moves in response to forces within and/or on the catheter, without structure allowing for manual manipulation. The corewire slide of FIG. 6 would also have unchanged views corresponding with FIGS. 5E and 5F since these views are unaltered by the different lever of this embodiment. With respect to the embodiment of FIGS. 6A-6D, corewire 604 may slide distally or proximally based upon fluid pressure within the catheter or the expansion/collapse of a balloon near the distal end of the corewire. Referring to FIGS. 6A and 6B, proximal section 602 of a corewire 604 may have a bent portion, or lever, 606. Bent portion 606 may be used to restrict the distance corewire 504 may slide in the proximal or distal directions relative to tubular shaft 102. In particular, corewire slide 606 may have features, such as a plurality of tabs, to limit the range of motion of corewire 604. Referring to FIG. 6C a view of hub 608 and slide 610 depicts an embodiment where bent portion 606 of corewire 604 is entirely within the walls of slide 610. As depicted in the corresponding sectional view in FIG. 6D, tabs 614, 616 limit the lateral sliding movement of corewire 604 within the catheter. Additional tabs 618 may be used to maintain a generally central position of corewire 604 within slide 608. This embodiment of the hub can similarly incorporate a flow meter 520 as shown in FIG. 5C.

In general, the catheters shown in FIGS. 1-6 can comprise one or more marker bands and/or other imageable components which can be used to position the balloon and profusion port(s) distal to the clot. While the structures provide appropriate constraints on the placement of imageable elements based on achieving desired mechanical performance, there generally is significant design flexibility for the placement of such radiopaque elements, and a person of ordinary skill in the art can manage such placement to achieve convenience for the corresponding procedures.

Catheter components can be formed from one or more biocompatible materials, including, for example, metals, such as stainless steel or alloys, e.g., Nitinol®, or polymers such as polyether-amide block co-polymer (PEBAX®), nylon (polyamides), polyolefins, polytetrafluoroethylene, polyesters, polyurethanes, polycarbonates, polysiloxanes (silicones), polycarbonate urethanes (e.g., ChronoFlex AR®), mixtures thereof, or other suitable biocompatible polymers. Radio-opacity can be achieved with the addition of metal markers, such as platinum-iridium alloy, tantalum, tungsten, gold, platinum-tungsten alloy or mixtures thereof, such as in the form of wire or bands, or through radio-pacifiers, such as barium sulfate, bismuth trioxide, bismuth subcarbonate, powdered tungsten, powdered tantalum or the like, or combinations thereof, added to the polymer resin. Generally, different sections of aspiration catheter can be formed from different materials from other sections, and sections of aspiration catheter can comprise a plurality of materials at different locations and/or at a particular location. In particular, it may be desirable to form seal components, the balloon, and/or the polymer valve from an elastomeric polymer, such as suitable polyurethanes, rubber, synthetic rubbers polyisoprene, ChronoPrene® (thermoplastic polyolefin elastomer), polydimethyl siloxane, polytetrafluoroethylene, other elastomers or combinations thereof. ChronoPrene® can be desirable due to its biocopatability, high flexural modulus, high elongation, high tensile strength and easy molding. In addition, selected sections of the catheter can be formed with materials to introduce desired stiffness/flexibility for the particular section of the catheter. Similarly, fittings can be formed form a suitable material, such as one or more metals and/or one or more polymers.

In some embodiments, a balloon/infusion catheter or appropriate portions thereof comprises a thermoplastic polymer with embedded metal elements, which reinforces the polymer. The wire can be braided, coiled or otherwise placed over a polymer tubing liner with some tension to keep the wire in place over the tubing liner. To decrease the chance of accidental removal of the radiopaque band from the catheter and to decrease the chance of the radiopaque band catching onto other objects within the vessel, a metal reinforcing wire can be used to cover or enclose the radiopaque band with the metal wire subsequently being embedded within the polymer. The metal wire can comprise interwoven wires, coil, combinations thereof, or the like. A polymer jacket can be placed over the metal wire, which is correspondingly covering the radiopaque band(s), and the heat bonding embeds the radiopaque marked band also. Placement of the marker band under metal wire can prevent the band from being separated from the catheter in the event that the wall is kinked or collapsed. If collapse or kinking of the catheter wall occurs, the braid-wire over the surface of the band collapses down over the marker band to prevent it from separating from the structure.

A reservoir of infusion fluid generally comprises a biocompatible liquid, such as sterile buffered saline, compatible blood, such as the patient's own blood or appropriately typed blood, or the like in a selected volume for the procedure, although a balloon inflation fluid source, in principle, can comprise a wider range of fluids if not used for infusion and maintained to not enter the patient. An infusion liquid can further comprise a drug or therapeutic, such as a clot dissolving drug, an antispasm drug, an oxygen carrier, stem cells, neuroprotective, anti-inflamatory, anti-apoptosis, growth factors, other stroke therapeutics or combinations thereof. Since the liquid is aspirated from the vessel, generally relatively large amounts of drug can be delivered for short time availability in the treatment region. Suitable clot dissolving drugs include, for example, tPA (tissue Plasminogen Activator), Urokinase, Tenecteplase, or the like. Neuro protectives can include, for example, Nerinetide, or NA-1, or Lumosa LT3001. The infusion reservoir can comprise a volume from about 0.1 cc (cubic centimeters) to about 50 cc, in further embodiments from about 0.15 cc to about 35 cc, and in additional embodiments from about 0.2 cc to about 25 cc. A person of ordinary skill in the art will recognize that additional ranges of volumes within the explicit ranges above are contemplated and are within the present disclosure.

### Treatment Systems and Hydraulic Clot Removal Assemblies

When the infusion balloon catheter is used in conjunction with an aspiration catheter, hydraulic forces can be delivered for clot removal. An additional medical tool may also be used to facilitate clot removal. In particular, an atherectomy device, such as a stent retriever can be used along with the hydraulic force generating components. For the delivery of aspiration, it can be desirable to use an aspiration catheter that connects with a guide catheter to use a portion of the guide catheter to provide a portion of the aspiration lumen. The aspiration and infusion catheters can work in conjunction to generate hydraulic and/or hydrodynamic forces, which can be referred to as hydraulic forces for convenience. Any other treatments, such as use of a stent retriever, generally, but not necessarily, would be performed prior to the application of hydraulic forces. The narrow profile of the balloon/infusion catheter described herein provides for the ability to perform such procedures with reasonable fluid flows in narrow vessels.

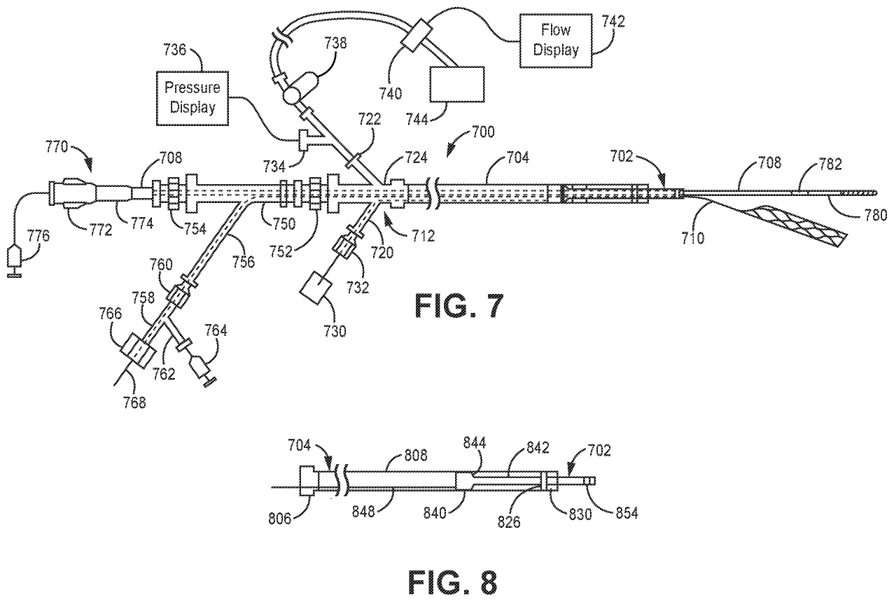

FIG. 7 depicts a view of an assembled embodiment of a treatment system with components to assist in a procedure. Specifically, treatment system 700 comprises an aspiration catheter 702 designed to interface with a guide catheter 704, and an balloon/infusion catheter 708 as well as a stent retriever 710 are inserted through lumen of guide catheter 704 and aspiration catheter 702. The components all interface through proximal fittings 710. A distal portion of the system is shown illustrating aspiration catheter 702 extending from guide catheter 704. A proximal portion of the treatment system shows proximal fittings 712 of the aspiration system extending proximally from guide catheter 704.

In embodiments, proximal fittings 712 have various branches providing desired functionalities as described in the several embodiments presented herein. In embodiments, a first branched manifold is connected at the distal end of guide catheter 704 with branches 720, 722, 724, which is shown as a three-branched manifold, although it can be provided as two Y-branch manifolds each with two branches connected in series. In this particular embodiment, first branch 720 is depicted with a tether 730 for stent retriever 710 exiting from a hemostatic valve 732. Second branch 722 is depicted with connections to an aspiration channel having a pressure sensor 734 connected to a pressure display 736, an inline filter 738 configured to remove any thrombus within the line to the negative pressure source, flow meter 740 connected to a flow display 742, and a negative pressure source 744. Suitable negative pressure sources include, for example, syringes, pumps, such as peristaltic pumps, piston pumps or other suitable pumps, aspirator/venturi, or the like, and commercial pumps are available for this purpose, such as Gomco Aspiration Pumps available from MIVI Neuroscience.

Extended hemostatic fitting 750 is connected with third branch 724 at connector 752, and terminates with a hemostatic valve 754. Extended hemostatic fitting 750 can have a branch 756 that interfaces with a docking branched manifold 758 at hemostatic valve 760. Docking branched manifold 758 may have a branch 762 connected to a fluid source 764 and a distal hemostatic valve 766 through which a tether 768 for aspiration catheter 702 can exit the closed catheter environment. The proximal end of aspiration catheter can dock with the docking manifold to provide for removal of aspiration catheter 702 through hemostatic valve 760 for rapid clearing of any clots from the aspiration catheter allowing its return to the vasculature.

As shown in FIG. 7, balloon/infusion catheter 708 exits through hemostatic valve 754. Balloon/infusion catheter 708 has a proximal hub 770 comprising a connector 772 and slide 774. Fluid source 776 may supply fluid to balloon/infusion catheter. Balloon/infusion catheter 708 can be one of the devices associated with FIGS. 1-6. Application of fluid through the single lumen catheter 748 may be used to inflate balloon 780 and provide infusion through infusion port 782.

Applicant has developed an aspiration catheter that interfaces with a guide catheter to form an aspiration lumen that extends through the aspiration catheter and partly through the guide catheter lumen. For application of aspiration, the proximal end of the aspiration catheter lumen is within the guide catheter lumen as depicted in FIG. 7 with a tether leading out from the patient to allow for moving the aspiration catheter to a desired location through manipulation of the tether. (Q-Catheter™, Mivi Neurosciences, Inc.).

An embodiment of the Q-Catheter™ is shown in FIG. 8, which involves separated components from FIG. 7. Referring to FIG. 8, guide catheter 704 is interfacing with aspiration catheter 702. The suction adapted guide catheter 702 comprises proximal connector 806 and tubular shaft 808. Proximal connector can connect to fittings with selected manifolds, such as shown in FIG. 7.

Tubular shaft 808 can have an approximately constant diameter along its length, or the guide catheter can have sections with different diameters, generally with a smaller diameter section distal to a larger diameter section. Tubular shaft 808 can have one or more radiopaque marker bands to facilitate positioning of the tubular shaft within the patient, and FIG. 8 shows a marker band 828 near the distal end of tubular shaft 808, although additional positions and/or alternative positions can be used as desired. At or near the distal end of the shaft, a stop 830 can be positioned to retain a portion of suction extension 704 within the lumen of tubular shaft 808.

Aspiration catheter 702 can comprises a proximal portion 840, suction tip 842, connection portion 844, and control structure 848, such as a control wire. All or a part of proximal portion 840 can be configured to remain within the lumen of guide catheter 704 while aspiration is applied. Proximal portion 840 can engage the interior of guide catheter 704 sufficiently to avoid significant flow between the guide catheter wall and proximal portion 840, while still allowing for relative movement of aspiration catheter 702 within guide catheter 704. Suction tip 842 is shown with radiopaque marker band 854 near the distal tip of suction tip 842, although again suction tip 842 can comprise a plurality of radiopaque marker bands if desired. Connection portion 844 connects proximal portion 840 and suction tip 842, which can be a transition portion that gradually changes diameter or a connector that forms a seal between the proximal portion and suction tip. In some embodiments, stop 830 is configured to engage an edge or other limiting structure of proximal portion 840. Control structure 848 can be a control wire or the like that connects with proximal portion 840 and extends exterior to the catheter. Control structure 848 can be used to control positioning of proximal portion 840 within the lumen of tubular shaft 808. Control structure 848 can comprise a control tool, such as a handle, slide or other the like that can anchor a control wire or other connecting element to facilitate movement of the control wire.

The guide catheter can have an outer diameter from about 5.5 Fr (1.667 mm diameter) to about 10 Fr (3.333 mm diameter), in further embodiments from about 6 Fr (1.833 mm diameter) to about 9 Fr (3 mm diameter), and in some embodiments from about 6.25 Fr (2 mm diameter) to about 8.5 Fr (2.833 mm diameter). The guide catheter measurement are generally referenced to the outer diameter, and the inner diameter is less than the outer diameter by twice the wall thickness. The length of the guide catheter can be from about 30 cm to about 150 cm, in further embodiments from about 35 cm to about 130 cm and in additional embodiments from about 40 cm to about 120 cm. The length of aspiration catheter 704 can be from about 30 cm to about 150 cm, in further embodiments from about 35 cm to about 130 cm and in additional embodiments from about 40 cm to about 120 cm. The outer diameter at the tip of the aspiration catheter generally is (diameter in mm=(Fr value)/3, Fr represents the French catheter scale) at least about 0.5 Fr less than the outer diameter of the distal section of the guide catheter. The smaller diameter of the tubular extension can provide access to desirable vessels, such as cerebral vessels. It was previously discovered that good suction properties could be obtained with a suction catheter with a stepped down diameter in a distal section. Thus, for example, the majority of the length of the aspiration catheter can be 6 Fr outer diameter while a distal section may be 5 Fr outer diameter, which roughly corresponding decreases in the inner diameters. A person of ordinary skill in the art will recognize that additional ranges of dimensions within the explicit ranges above are contemplated and are within the present disclosure.

Further options and details regarding the aspiration catheter in FIG. 8 can be found in U.S. Pat. No. 10,716,915 to Ogle et al., entitled “Catheter Systems for Applying Effective Suction in Remote Vessels and Thrombectomy Procedures Facilitated by Catheter Systems,” and U.S. Pat. No. 10,478,535 to Ogle, entitled “Suction Catheter Systems for Applying Effective Aspiration in Remote Vessels Especially Cerebral Arteries,” both of which are incorporated herein by reference. Desirable fittings for use with these aspiration systems including a docking structure are described in published U.S. patent applications 2019/0183517 to Ogle, entitled “Suction Catheter Systems for Applying Effective Aspiration in Remote Vessels Especially Cerebral Arteries,” and 2021/0228844 to Ogle, entitled “Suction Catheter Systems with Designs Allowing Rapid Clearing of Clots,” both of which are incorporated herein by reference.

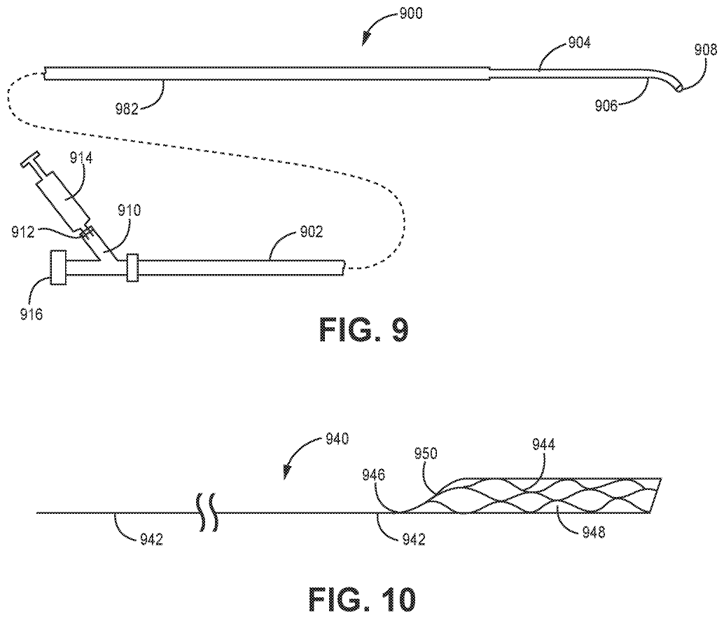

Various other aspiration catheters have been developed for providing improved suction within the narrow tortuous vessels of the cerebral vasculature. In some embodiments, these aspiration catheters have a single catheter structure that extends through the full length of a guide catheter and can have a narrowed distal tip that can reach into narrow vessels but provide high flows out of the vessel due to the larger proximal lumen. These designs are described in U.S. Pat. No. 9,662,129 to Galdonik et al., entitled “Aspiration Catheters for Thrombus Removal,” incorporated herein by reference. Referring to FIG. 9, aspiration catheter 900 for accessing smaller vessels comprises a tube 902, a reduced diameter distal segment 904 with an average diameter smaller relative to the average diameter of the tube, an optional curved distal tip 906, a radiopaque marker band 908, which may be at or near the distal tip whether or not curved, a proximal fitting 910, an aspiration connection 912, a negative pressure source 914, and a proximal port 916 for insertion of guide structures or other devices through the catheter lumen. Aspiration catheter 900 can optionally have a rapid exchange configuration with a rapid exchange port.

Distal segment 904 can have an outer diameter from about 25 percent to about 95 percent of the average outer diameter of tube 902 of the catheter, and in further embodiments from about 45 to about 90 percent and in additional embodiments from about 60 to about 85 percent of the average diameter of the tube. For example, distal segment 904 can have an outer diameter range from about 0.015 to about 0.120 inches, and tube 902 can have an outer diameter range from about 0.030 to about 0.150 inches, in other embodiments from about 0.040 to about 0.125 inches and in further embodiments from about 0.045 to about 0.120 inches. A person of ordinary skill in the art will recognize that additional ranges of dimensions within the explicit ranges above are contemplated and are within the present disclosure. In optional embodiments, a bent or curved tip can provide improved tracking during delivery into a patient's vessel by controlling tracking along a guide structure extending from the tip.

Stent retrievers are commercially available for acute ischemic stroke treatment. For example, stent retrievers are available from Medtronic Corp. (Solitaire®,) and Stryker (TrevoProvue™). Stent retrievers are described, for example, in U.S. Pat. No. 8,795,305 to Martin et al., entitled “Retrieval systems and methods of use thereof,” incorporated herein by reference. Desirable thrombus engagement devices are described in U.S. Pat. No. 10,463,386 to Ogle et al., entitled “Thrombectomy Devices and Treatment of Acute Ischemic Stroke With Thrombus Engagement,” incorporated herein by reference. An example of a stent retriever is shown in FIG. 10.

Referring to FIG. 10, a stent retriever 940 is shown comprising a tether 942, a metal frame 944, and connector 946. The body of metal frame 944 generally is cylindrical if it is unconstrained with openings 948 between elements of the metal frame, and the cylinder can have a proximal transition region 950 that open ups toward the connector 946, which as attached at a point along the cylinder edge. Connector 946 can be a weld of metal frame elements to the tether 942 and/or can comprise a mechanical fastener, such as a metal band which can be radiopaque.

Tether 942 can be a wire, coil or the like. Tether 942 generally can have a length from about 30 cm to about 300 cm, in further embodiments from about 40 cm to about 250 cm, and the length may be selected depending on the specific procedure and vessel through which the device is introduced. Tether can have a diameter from about 0.001 inches to about 0.014 inches, in further embodiment from about 0.002 inches to about 0.01 inches and in additional embodiments from about 0.003 inches to about 0.008 inches. A person of ordinary skill in the art will recognize that additional ranges of tether dimensions within the explicit ranges above are contemplated and are within the present disclosure.

The treatment system is generally appropriately sterilized, such as with e-beam or gas sterilization. The treatment system components can be packaged together or separately in a sealed package, such as plastic packages known in the art. The package will be appropriately labeled, generally according to FDA or other regulatory agency regulations. The treatment system can be packaged with other components, such as a guidewire, microcatheter, and/or other medical device(s). The packaged system generally is sold with detailed instructions for use according to regulatory requirements. As used herein, guidewire or guide structure can refer to any appropriate elongated element suitable to guide the delivery of the treatment catheter, such as a wire, coil, or integrated guide structure with a core element and an overtube. If the balloon/infusion catheter is delivered analogously to a guidewire, a separate guidewire may not be used for the procedure.

### Procedures for Clot Removal

In general, the balloon/infusion catheters described herein can be used in any suitable medical procedure, and the system components that can be used with the balloon/infusion catheter can be selected appropriately. As noted above, the balloon/infusion catheter can be particularly effective for clot removal from vessels, and there is a particular significance for clot removal for acute ischemic stroke treatment. The following discussion focuses on ischemic stroke treatment, and a person of ordinary skill in the art will appreciate the ability to generalize this discussion for other indications.

For the performance of an acute ischemic stroke procedure, a guide catheter is generally put into place near the beginning of the procedure to establish access into the carotid artery. Various approaches can be used for positioning the devices then in the cerebral arteries through the guide catheter. The balloon/infusion catheter described herein is designed with excellent maneuverability and a low profile. So this catheter can be delivered like a guidewire from the guide catheter to position the balloon/infusion catheter with the balloon and infusion port distal to the clot. The aspiration catheter can then be tracked over the balloon/infusion catheter to its desired position. The balloon on the balloon/infusion catheter can be inflated and the procedure commenced. In an alternative embodiment, a separate guidewire can be used to establish a position past the clot. A microcatheter can be tracked over the guidewire, and the guidewire can be removed once the microcatheter is in place. The balloon/infusion catheter can be delivered through the microcatheter, and the microcatheter can then be moved and the aspiration catheter delivered. The following more detailed discussion focuses on the embodiment in which the balloon/infusion catheter is delivered like a guidewire. The alternative procedure is described in in the '778 application cited above and can be adapted for the improved balloon/aspiration catheter described herein.

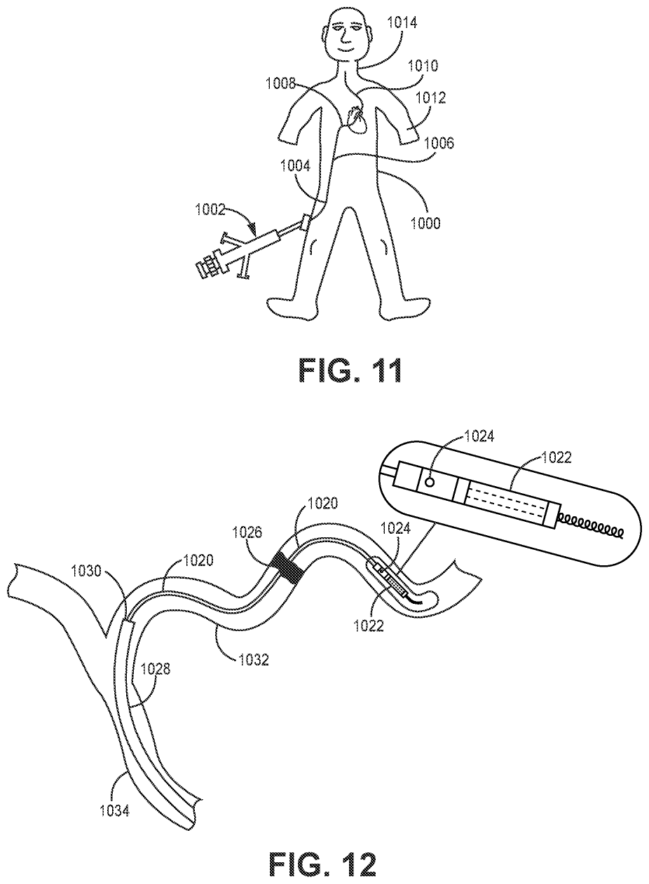

Referring to FIG. 11, a human patient 1000 is shown with appropriate portions of a treatment system 1002 inserted into their femoral artery 1004 where components are guided up the descending aorta 1006 around the aortic arch to the ascending aorta 1008 where components are guided into a carotid artery 1010 (left or right) prior to reaching the heart. The distal end of the components are then guided through the patient's neck into an internal carotid artery and then into the cerebral arteries forming the neurovasculature. While this can be a desirable approach to the cerebral arteries, alternative access locations include the arm 1012 or the neck 1014. Radiopaque markers generally are used to assist with placement of the various devices including placement of the balloon and infusion port(s) distal to the clot using real time imaging.

The basic arrangement for positioning the components for the performance of the procedure is shown schematically in FIG. 12. As noted above, the basic concept is to generate hydraulic forces between an expanded occlusion balloon and the aspiration catheter to dislodge the clot. Preliminary preparations for the percutaneous procedure can comprise access into the arterial system along with placement of hemostatic fittings and other appropriate components providing access into the patient. In some embodiments, access is obtained into a femoral artery, although other access locations can be used. The guide catheter can have a balloon, and whether or not the guide catheter has an occlusion balloon, the guide catheter may or may not be used for suction.

Common features of the procedures described herein comprise obtaining access to the cerebral artery distal to a clot, which is generally performed with a guide structure. The balloon/infusion catheter described is designed to act as a guide structure so that a guidewire for accessing the cerebral artery may not be needed. A position distal to the clot is generally maintained until the clot is removed at least in part. Once a guide structure has established position, the components can be delivered over the guide structure. At some point, the balloon/infusion catheter is delivered to position the balloon distal to the clot and infusion port(s) between the balloon and the clot. A microcatheter may or may not be involved in the delivery process. Also, an aspiration catheter can be placed prior to initiation of hydraulic forces. Using fluid flow, the clot or portions thereof are removed from the vessel. The components of the treatment system can be removed using a reasonable procedure designed to avoid release of emboli. While this order of steps accounts for practical implementation and provides an overview of the procedure, reasonable variation in the order of steps can be used if consistent with the overall procedure. Thus, appropriate steps may be performed in a different order, and some steps can be performed in substeps that may be interspersed with portions of other steps. Also, repositioning of various components can take place through the procedure as appropriate and desired by the user.

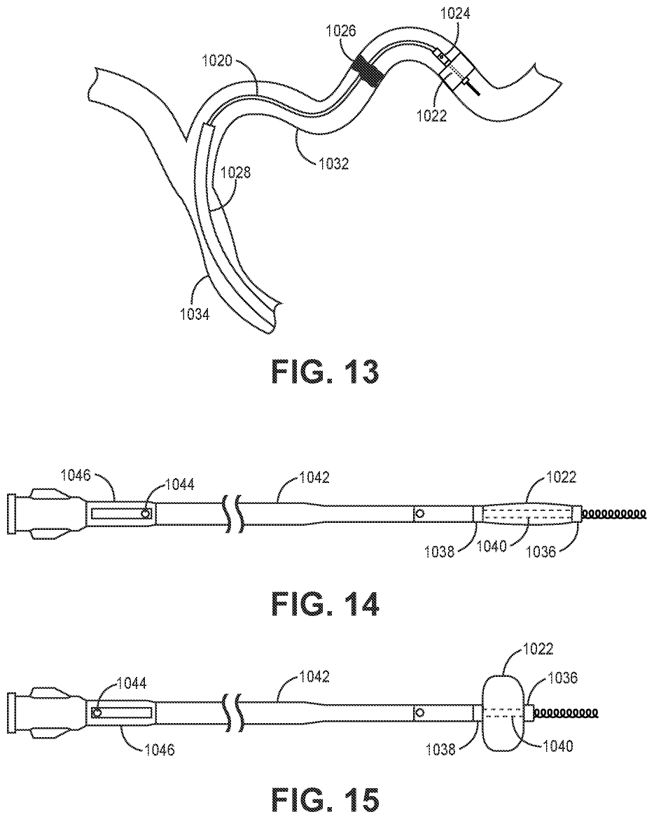

Referring to FIG. 12, a balloon/infusion catheter 1020 is inserted in the artery to place balloon 1022 and infusion port(s) 1024 past clot 1026. In some procedures, aspiration catheter 1028 can be positioned with its distal tip 1030 entering cerebral vessel 1032. Depending on the specifics of the vasculature and the aspiration catheter design, aspiration catheter distal tip 1030 may be brought closer or further from clot 1026, and the aspiration catheter can be in an upstream cerebral artery 1034 at an appropriate position. If balloon/infusion catheter 1020 is used as a guide structure the distal portion of aspiration catheter 1028 can be tracked into position over the balloon/infusion catheter after it is in position. Referring to FIG. 13, balloon 1022 is inflated to inhibit flow in either direction past the balloon. Aspiration catheter 1028 could also be a Balloon Guide Catheter (BGC) or used in conjunction with a BGC.

Aspiration and infusion generally are initiated following inflation of balloon 1022. Also, aspiration and infusion can be initiated approximately simultaneously or sequentially with a planned time spacing, and even if planned to be approximately simultaneous, the processes are generally separately initiated so that a slight delay generally results from the time to start the processes. In practice, a medical professional may develop a technique according to personal preferences with respect to the order of inflating the balloon, starting aspiration, and starting infusion. For example, a professional may want to start aspiration, and then inflate the balloon followed by infusing liquid. Generally, any reasonable process order can be used with appropriate attention to avoiding the flow of emboli downstream in the vessel based on release of thrombus from the clot. Further, additional devices, such as atherectomy devices, may be introduced to facilitate clot removal, as described further below.

As depicted in FIGS. 14 and 15, when balloon 1022 expands, a distal end 1036 of the balloon 1022 is drawn closer to a proximal end 1038 of the balloon. Accordingly, the corewire 1040 within balloon 1022 and shaft 1042 slides in the proximal direction during balloon 1022 expansion, and, by extension, may slide in the distal direction when balloon 1022 retracts. As discussed above corewire 1040 has a bent portion 1044 that moves between tabs within slide 1046 which limit the distance corewire 1040 may travel. In embodiments, bent portion 1044 may extend beyond slide 1046 allowing for manual manipulation. Moving the bent portion 1044 proximally or distally may cause balloon 1022 to respectively expand or retract. When corewire 1040 is moved while fluid is introduced or removed through a lumen in shaft 1042, an operator may exert some control over the speed with which balloon 1022 expands or compresses by controlling the rate of fluid delivery.

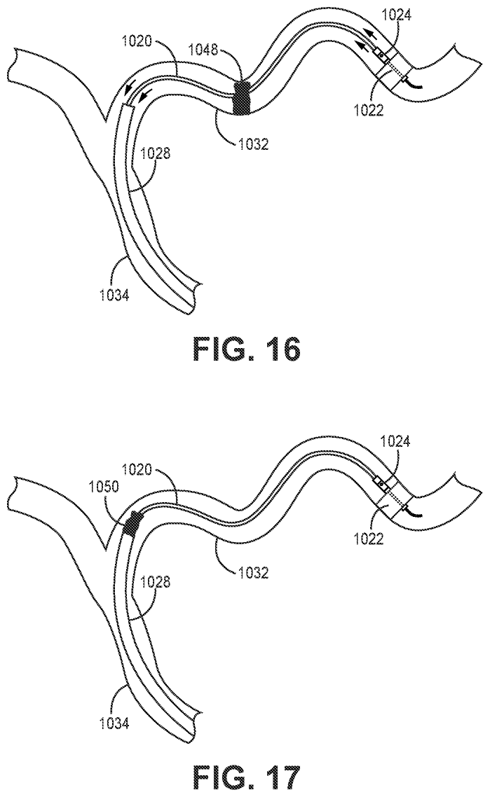

Referring to FIG. 16, aspiration is depicted with flow arrows near the aspiration opening into aspiration catheter 1028 and flow arrows near infusion port 1024 indicate infusion into the vessel of infusion liquid. Due to the occlusion effect of the clot, the infusion of liquid initially tends to increase the pressure between the clot and the balloon. A pressure increase tends to increase the vessel diameter in response if the vessel wall has some elasticity. The infusion pressure and liquid volume can be controlled to avoid damage to the vessel. The amount of liquid delivery by infusion may optionally change as the clot is dislodged and liquid can flow more readily to the aspiration catheter. Regardless, the hydraulic pressure established by the aspiration from the proximal position and infusion from a distal position established hydraulic forces moving in a distal to proximal direction.

If the system is configured with pressure and/or flow sensors as described above, the information on the pressure and/or flow can help to guide the procedure. For example, the measurements can indicate whether or not the aspiration catheter is blocked of kinked. A blockage can contraindicate delivery of contrast dye or other fluids through the aspiration catheter to avoid reinsertion of thrombus. Also, if aspiration flow has stopped, it can be desirable to stop delivery of infusing liquid from balloon/infusion catheter until aspiration issues have been resolved, to avoid excess pressures in the vessel, which can be separately monitored. Various alarms can be associated with the pressure/flow measurements to alert the medical professional to potentially dangerous conditions. In general, the medical professional performing the procedure can use the pressure and/or flow readings in any convenient way.

Referring to FIG. 16, due to the hydraulic forces in cerebral vessel 1032, thrombus 1048 from clot 1026 moves in a proximal direction. Continued movement of thrombus results in captured thrombus removed by aspiration catheter 1028 and/or captured thrombus 1050 at the opening into aspiration catheter 1028, as shown in FIG. 17. Depending on the size of the clot and rigidity of the clot material, the clot may or may not completely enter into the lumen of the aspiration catheter. Thrombus may fragment during the removal process with a portion of thrombus from clot 1026 removed through aspiration catheter 1028 and a portion of thrombus captured at the opening of aspiration catheter 1028 or generally any amounts on the continuum of all of the thrombus removed by aspiration or all of the thrombus collected on the tip of aspiration catheter 1028. Infusion flow can be reduced or turned off if aspiration flow diminishes due to occlusion or partial occlusion of the aspiration catheter by the clot.

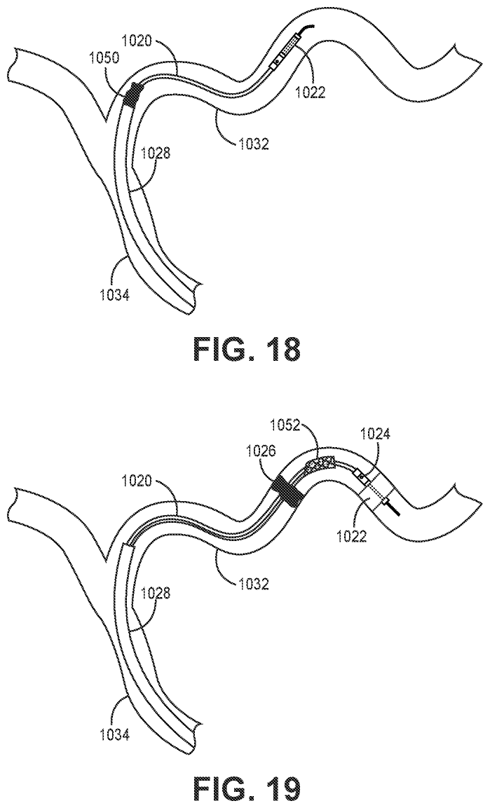

Following appropriate capture of thrombus, balloon 1022 can be deflated, partially or approximately fully deflated, and then removed from the vessel. If manual control is available with the balloon/infusion catheter design, the corewire can be moved in a proximal direction to facilitate balloon deflation. Referring to FIG. 18, balloon 1022 is deflated, and removal from cerebral artery 1032 has been initiated. Movement of the balloon/infusion catheter in a proximal direction can tend to move the corewire in a distal direction relative to the catheter tube due to drag against the fluid, and this movement tends to flatten down the deflated balloon.

Aspiration may or may not be maintained during the device removal process or separate portions thereof at the same pressure or a reduced pressure or combination thereof at different times. Infusion is generally stopped prior to deflating balloon 1022. Aspiration catheter 1028 can be removed simultaneously with balloon/infusion catheter 1020 or following removal of balloon/infusion catheter 1020. In some embodiments, aspiration catheter 1028 is maintained in position until balloon 1022 is withdrawn close to the opening into aspiration catheter 1028 following which catheters 1028, 1020 are removed together. In general, a selected order for removing aspiration catheter 1028 and balloon/infusion catheter 1020 can be selected in various reasonable orders and variants thereof with some consideration that medical professionals may develop preferences based on their experiences and further clinical studies may suggest specific nuances of the procedures. The order of removal of the components can be selected to facilitate removal of the thrombus with a low risk of embolization from the thrombus. Ultimately, all of the devices of the system are removed from the patient and the entry point into the patient is closed.

Referring to FIG. 19, in some embodiments, a stent retriever 1052 may be positioned between clot 1026 and balloon 1022. Stent retriever 1052 can be deployed to loosen clot 1026 or portions thereof in conjunction with the hydraulic force application. The stent retriever can be removed with the aspiration catheter at the end of the procedure.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated.

What is claimed is:

1. A balloon/infusion catheter comprising:
a corewire comprising a shaft, a distal landing structure and a proximal lever;
a tube comprising a shaft with a single lumen, a proximal end, and a distal end, and one or more infusion ports near the distal end, wherein the corewire extends through the tube and wherein a gap between the corewire and a wall of the tube allows fluid to flow through the lumen;
a balloon comprising a polymer sleeve in a sealing engagement at a first end with the tube and at a second end with the landing structure such that a sealed enclosure is formed spanning between the distal end of the tube and the landing structure; and
a proximal hub attached around the proximal end of the tube in a sealed configuration, the proximal hub comprising a wall, a corewire slide and a connector forming a continuous lumen through the hub extending the lumen of the tube, wherein the corewire slide comprises a distal stop and a proximal stop that are configured to engage the proximal tab of the corewire at corresponding positions of the corewire relative to the tube to limit the range of relative positions of the corewire with the corewire free to slide within the tube between the proximal stop and the distal stop, and wherein the connector establishes a fluid channel from an opening into the connector to the interior of the balloon and to the one or more infusion ports.

2. The balloon/infusion catheter of claim 1 wherein the proximal lever comprises a bent section of a flattened length of the corewire.

3. The balloon/infusion catheter of claim 1 wherein the distal stop and the proximal stop each comprise a flange extending into the lumen of the proximal hub from an intact wall of the hub.

4. The balloon/infusion catheter of claim 1 wherein the corewire slide comprises a slot through the wall of the hub with the proximal tab of the corewire extending through the slot, wherein the slot and proximal tab are covered with a polymer cover to form a liquid tight seal, wherein a flange limits movement of the corewire to maintain the proximal tab in the slot and wherein the dimensions of the slot provide limits on the relative position of the corewire relative to the tube.

5. The balloon/infusion catheter of claim 1 wherein the proximal lever of the corewire does not extend through the wall of the hub.

6. The balloon/infusion catheter of claim 1 wherein the balloon in an initial uninflated state is a cylindrical, non-prestretched elastomer.

7. The balloon/infusion catheter of claim 1 wherein a polymer valve covers the infusion ports, wherein the polymer valve opens in response to an overpressure with respect to the pressure differential between the lumen of the tube relative to the pressure external to the tube

8. The balloon/infusion catheter of claim 7 wherein the balloon expands at fluid pressured in the lumen lower than pressures to induce the valve opening.

9. The balloon/infusion catheter of claim 1 wherein the corewire further comprises a distal coil positioned distal to or overlapping with the distal landing structure.

10. The balloon/infusion catheter of claim 9 wherein the distal coil is connected to the corewire at a solder joint extending from about 0.5% to 25% of the coil length.

11. The balloon/infusion catheter of claim 1 wherein the corewire comprises a tapered section connecting a smaller diameter segment with a larger diameter segment.

12. The balloon/infusion catheter of claim 1 wherein the corewire comprises two or more outwardly extending wings along at least a portion of its length to limit radial movement of the corewire within the shaft of the tube.

13. The balloon/infusion catheter of claim 1 wherein the tube has a distal annular opening from the lumen around the corewire into the interior of the balloon and wherein the connector of the hub is in fluid communication with a fluid reservoir.

14. The balloon/infusion catheter of claim 13 wherein the fluid reservoir is associated with an infusion pump suitable to deliver fluid at a set pressure or flow rate.

15. The balloon/infusion catheter of claim 1 wherein the tube further comprises a pressure sensor configured to read a pressure external to the tube.

16. The balloon/infusion catheter of claim 1 wherein the corewire comprises a optical fiber pressure sensor that is exposed in the region interior to the balloon.

17. The balloon/infusion catheter of claim 1 wherein the hub comprises a flow meter.

18. A system for clot removal from a bodily vessel comprising:
the balloon/infusion catheter of claim 1;
a guide catheter; and
an aspiration catheter, wherein the aspiration catheter can be configured to pass through the guide catheter with a distal section with an aspiration port extending distally from the guide catheter and wherein the balloon infusion catheter can pass through the length of the aspiration catheter to have the balloon and one or more infusion ports extending distally from the aspiration catheter.

19. A method for performing fluid infusion into a sealed off segment of a blood vessel, the method comprising:
guiding balloon/infusion catheter having a single lumen, without the aid of a guidewire from the carotid artery to a cerebral artery, wherein the balloon/infusion catheter comprises a tube with one or more infusion ports extending through the wall of the tube, a hub with a connector secured to the proximal end of the tube, a polymer balloon sealed on one side to the tube distal to the polymer valve, a corewire extending through the lumen of the tube, a landing structure secured to another end of the balloon such that a sealed enclosure is formed spanning the distal end of the tube and the landing structure, wherein the landing structure is secured to the corewire, and slide limiter to limit the relative motion of the corewire relative to the tube, wherein during the guiding, the balloon is in a taut configuration over an opening into the lumen and wherein the hub is connected to a fluid source for inflating the balloon and providing infusion liquid;
inflating the balloon to seal off the blood vessel, wherein the inflation comprises delivering fluid into the lumen; and
infusing fluid proximal to the inflated balloon by delivering sufficient pressure of fluid into the lumen to release fluid through the polymer valve.

20. The method of claim 19 further comprising positioning both the balloon and perfusion ports distal to a clot.

21. The method of claim 19 wherein the corewire comprises a coil at the distal end of the corewire.

22. The method of claim 19 further comprising monitoring a pressure and/or a flow rate of the proximal fitting.

23. The method of claim 19 further comprising delivering the balloon/infusion catheter through a proximal fitting of a guide catheter and through an aspiration catheter.

24. The method of claim 23 wherein aspiration is applied for at least a portion of the time over which liquid is infused such that hydraulic forced are established in the vessel.

25. The method of claim 23 wherein the aspiration catheter has a proximal portion that engages the inner wall of the guide catheter, a suction extension that extends past the distal end of the guide catheter, and a tether that extends past the proximal end of the guide catheter to establish a suction lumen that extends form a negative pressure source through a portion of the guide catheter and through the aspiration catheter.

26. The method of claim 19 wherein a polymer valve covers the one or more infusion ports and wherein the polymer valve opens when the pressure is sufficient that the balloon is inflated.

27. A balloon/infusion catheter comprising:
a corewire;
a tube comprising a shaft with a single lumen, a proximal end, and a distal end, and one or more infusion ports near the distal end, wherein the corewire extends through the tube and wherein a gap between the corewire and a wall of the tube allows fluid to flow through the lumen;
a balloon having a sealed interior in fluid communication with the single lumen;
a proximal hub attached around the proximal end of the tube in a sealed configuration, the proximal hub comprising a wall and a connector forming a continuous lumen through the hub extending the lumen of the tube and wherein the connector establishes a fluid channel from an opening into the connector to the interior of the balloon and to the one or more infusion ports; and
a flow meter, wherein the flow meter is configured to provide a value related to the flow rate through the single lumen.

28. The balloon/infusion catheter of claim 27 wherein a corewire comprises a shaft, a distal landing structure and a proximal lever, wherein the balloon comprises a polymer sleeve in a sealing engagement at a first end with the tube and at a second end with the landing structure such that a sealed enclosure is formed spanning between the distal end of the tube and the landing structure, and wherein the hub further comprises a corewire slide, the corewire slide comprising a distal stop and a proximal stop wherein the distal stop and proximal stop are configured to the proximal tab of the corewire at corresponding positions of the corewire relative to the tube to limit the range of relative positions of the corewire with the corewire free to slide within the tube between the proximal stop and the distal stop.

29. The balloon/infusion catheter of claim 27 wherein the flow meter comprises an optical fiber with a Bragg grating and an LED within the hub.

30. The balloon/infusion catheter of claim 27 wherein the flow meter comprises a Doppler ultrasound sensor mounted near the distal end of the balloon/infusion catheter on the hub or adjacent the hub.
